SEQUENCE LISTING (2 sequences)

SEQ ID NO: 1 - DNA, 15 bp, Artificial
  Synthetic primer
ngaayyttcnngaay

SEQ ID NO: 2 - DNA, 15 bp, Artificial
  Synthetic primer
agaacgttctagaac

(19) United States
(12) Patent Application Publication
(10) Pub. No.: US 2009/0022785 A1
(43) Pub. Date: Jan. 22, 2009

(54) Permeable Capsules

(75) Inventor: Thomas Czerny, Vienna (AT)

Correspondence Address: FULBRIGHT & JAWORSKI L.L.P., 600 CONGRESS AVE., SUITE 2400, AUSTIN, TX, 78701, US

(73) Assignee: VETERINARMEDIZINISCHE UNIVERSITAT WIEN, Vienna (AT)

(21) Appl. No.: 11/913,491

(22) PCT Filed: May 3, 2006
(86) PCT No.: PCT/AT2006/000183

§ 371 Date: Nov. 2, 2007

(30) Foreign Application Priority Data
May 3, 2005 (AT) A 762/2005

(51) Int. Cl. A61K 48/00; A61K 9/48

(52) U.S. Cl. 424/451; 424/93.21

(57) ABSTRACT

The present invention relates to permeable capsules comprising at least one cell comprising a recombinant nucleic acid molecule with a heat inducible promoter operably linked to a nucleic acid encoding for a protein, a peptide or a functional nucleic acid molecule and at least one heat emitting agent capable to emit heat when exposed to electromagnetic radiation or to a magnetic field.

This application is a national phase application under 35 U.S.C. 371 of International Application No. PCT/AT2006/000183 filed 3 May 2006, which claims priority to Austrian Application No. A 762,2005 filed 3 May 2005. The entire text of each of the above-referenced disclosures is specifically incorporated by reference herein without disclaimer.

The present invention relates to permeable capsules usable in cell therapy.

In recent years several different methods to treat patients suffering from diseases, which, for instance, are mediated by genetic defects, were developed. The most promising therapies include gene and cell therapy.

In the course of a gene therapy, genes are inserted into the cells of patients by direct or indirect routes, where the inserted genes can be integrated into the chromosome of the target cell. The object of gene therapy is therefore to add, repair or block the expression of genes.

In an essential step of the gene therapy the cloned genes have to be introduced and expressed in the cells of a patient in order to overcome a specific disease. The gene(s) of interest may be transferred into a target cell ex or in vivo. Ex vivo gene transfer initially involves the transfer of genes into cells grown in culture. Those cells which have been transformed successfully are selected, propagated by cell culture in vitro and then introduced into the individual. In contrast thereto, in vivo gene transfer involves the transfer of cloned genes directly into the tissues or cells of the patient. This may be the only possible option in tissues where individual cells cannot be cultured in vitro in sufficient numbers (e.g. brain cells) and/or where cultured cells cannot be re-implanted efficiently into patients.

In the prior art numerous different physico-chemical and biological methods that can be used to transfer genes into human cells are disclosed. One suitable method involves the use of liposomes, which mimic the structure of biological membranes. The nucleic acid molecule to be transferred is packaged in vitro with the liposomes and used directly for transferring said nucleic acid to a suitable target tissue in vivo. The lipid coating allows the nucleic acid molecule to survive in vivo, to bind to cells and to be endocytosed into the cells. However, the efficiency of gene transfer is low and the introduced nucleic acid molecule is normally not designed to integrate into the chromosomal DNA. Alternatively, particle bombardment by using a gene gun may be employed. This method involves the initial coating of a nucleic acid molecule to metal pellets, which are afterwards fired from a gun into the target cells. However, the most promising and the most effective vehicles to transfer a defined nucleic acid molecule into a cell are vectors, preferably viral vectors. Mammalian viral vectors, like retroviral (e.g. murine leukemia virus) and adenoviral vectors, are the preferred vehicles for gene transfer because of their high efficiency of transduction into human cells.

However, gene therapy poses various risks, especially when vector systems are employed. Viral vectors in particular carry an assortment of risks. Viral vectors that are not properly targeted may infect a broader range of cells than intended. Beyond the risks associated with viral infection, the non-specific integration of genes presents the possibility of disrupting gene regulation in the host genome potentially leading to cancer. For example, the integration can cause activation of an oncogene or it could inactivate a tumor suppressor gene or a gene involved in apoptosis (programmed cell death). Another aspect of the safety of gene therapy is the fact that viral vectors may reach germline cells. If such an event occurs, the modified genes will become heritable.

In contrast thereto, cell therapy is used to replace diseased or dysfunctional cells with healthy, functioning ones. Furthermore, the introduced cells can provide additional functions, not normally active in the host. This technique is applied to a wide range of human diseases, including cancer, neurological diseases (e.g. Parkinson's disease), spinal cord injuries and diabetes. The cells used in cell therapy may be isolated either from animals or humans. However, individuals undergoing cell therapy treatments which use cells transplanted from animals or other humans run the risk of cell rejection. Also the risk of the cells to transmit bacterial or viral infections or other diseases and parasites to another individual has to be taken into account.

In order to overcome the safety concerns related to gene and cell therapy several methods and means have recently been developed. One advantageous method to introduce cells into a body is the encapsulation of said cells (Chang T M et al., Mol Med Today (1998), 4:221-227; Saitoh Y. et al., Cell Transplant (1995), 1:S13-17). Encapsulated cells may also be used advantageously as or in implants because the nutrients and the expressed gene products are able to pass the outer barrier of the implant unhindered, the cells are not exposed to the immune system and therefore they do not provoke any immune reaction and the implant can easily be introduced and removed by surgical techniques. For instance, encapsulated cells may be used in cell therapy for the post-operative treatment of cancer patients. Such cells secreting an anti-angiogenic substance (e.g. endostatin) are implanted in the proximity of the removed tumor in order to stop the growth of cells at that site (Bjerkvig R. et al., Acta Neurochir. Suppl. (2003), 88:137-141). Encapsulated cells may also be used as e.g. orally administered pharmaceuticals which pass the gastrointestinal tract and are excreted from the body within a certain period of time. The temporary presence in the body would alleviate safety concerns concerning genetically engineered cells (see e.g. Prakash S, et al. (2000) Int J Artif Organs. 23:429-35).

The expression of the proteins, peptides, metabolites and therapeutically active agents produced and secreted by encapsulated cells can be continuously directed by a constitutive promoter. More desireable is a regulated expression, which can be achieved by promoters which are induced by steroid hormones, isopropyl beta-D-thiogalactoside (IPTG), doxycyclin (Dox) or heavy metals. Most of these substances may induce serious side effects when administered to a mammalian host (see e.g. Saitoh Y. et al., Molecular Brain Research (2004), 121:151-155). After administration, these substances have to reach their target cells by diffusion, which is slow and difficult to control. Consequently, many of these promoters can not be regulated in a way allowing an effective dosage of the therapeutic agent and, hence, a satisfactory treatment of a disease.

The WO 99/06059 relates to methods and means for the administration of antioxidizing substances and conjugated fatty acids to mammalian cells in order to protect said cells from immunotoxicity and lipotoxicitiy. These substances can be produced by cells comprising plasmids allowing the expression of enzymes responsible for the biosyntheses of these substances.

In the DE 101 58 331 A1 PNA conjugates are described which allow to target the transport of a nucleic acid sequence, specifically to compartments of eukaryotic cells.

The EP 0 330 801 relates to the use of ferromagnetic, paramagnetic and diamagnetic particles in the diagnosis and treatment of diseases. In the course of such a treatment said particles are administered to a mammalian body and a magnetic field is applied to the site to be treated.

The U.S. Pat. No. 4,359,453 relates to the treatment of atherosclerosis by application of external electromagnetic energy capable of generating heat in intercellular particles within atherosclerotic plaques. Induction of heat in said plaques leads to their bioresolution.

It is an object of the present invention to provide means to be used in cell therapy, which are safe when applied to an individual and will allow the control of the production and of the release of proteins, peptides, functional nucleic acids, therapeutic substances and virus particles through the capsule barrier into the capsule-surrounding tissue or body fluid.

Therefore, the present invention relates to a permeable capsule comprising at least one cell comprising a recombinant nucleic acid molecule with a heat inducible promoter operably linked to a nucleic acid encoding for a protein, a peptide or a functional nucleic acid molecule and at least one heat emitting agent capable to emit heat when exposed to electromagnetic radiation or to a magnetic field.

According to the present invention “permeable capsule” relates to a capsule having a barrier surrounding the cell(s) to be encapsulated in order to reduce the mechanical stress on said cell(s) allowing free diffusion of nutrients to said cell(s) and of products secreted by said cell(s) through said barrier and blocking effective access by the host immune system. A stable capsule has to be substantially insoluble, biocompatible and non-reactive with the environment into which it will be introduced (e.g. implanted). Methods of encapsulation, which may be used to produce the capsules according to the present invention are known to the person skilled in the art (see e.g. Chang T M S, Nature Reviews Drug Discovery (2005) 4:221-235, Hauser O. et al. Current Opinion in Molecular Therapeutics (2004) 6:412-420; EP 0 835 137 B1, Sommer B. et al. Molecular Therapy (2002), 6:155-161; Goosen M. F. A. et al. Biotechnol. Bioeng. (1985), 27:146-150). For instance, the permeable capsule material may consist of polymers of alginate, polyacrylate or cellulose sulfate (see also Orive G. et al. Trends Biotechnol (2004), 22:87-92). Cells encapsulated in such permeable polymers are able to survive for a long period of time when implanted in a mammalian host. For instance, Sommer B. et al. (Molecular Therapy (2002), 6:155-161) determined the survival time to be at least 43 weeks (implant was removed after 43 weeks from a mammalian host).

According to the present invention said nucleic acid molecule may be either part of a vector, episome or chromosome. In order to guarantee a high stability of the recombinant nucleic acid molecule introduced in the host cell, said nucleic acid molecule is preferably integrated in the chromosome of said host cell.

According to the present invention the terms “heat inducible promoter” or “heat shock promoter” (both terms are synonyms) refer to nucleic acid sequences which, at a temperature rise from a lower temperature (e.g. normal physiological temperature; e.g. mammalian cells 36°-37° C.) to a higher temperature (e.g. for mammalian cells at least 39° C., preferably 40° C. or more, more preferably 41° C. or more) leads to an increased transcription rate of a nucleic acid fragment operably linked to said promoter. Therefore, the nucleic acid fragment is minimally or not transcribed at normal physiological conditions. If a basal-expression is detected at normal physiological conditions the transcription rate may increase at least 2 times, preferably at least 5 times, more preferably at least 10 times, in particular at least 100 times, upon induction of a heat inducible promoter. The characteristic features of “heat inducible promoters” are disclosed, for instance, in Morimoto R I et al. (J. Biol. Chem. (1992) 267:21987-21990).

However, heat shock promoters can similarly be activated by other conditions causing cellular stress like heavy metals, organic substances, amino acid analogues. In particular electromagnetic radiation can induce strong heat shock responses without substantially increasing the temperature in the cells (de Pomerai D. et al. (2000) Nature 405, 417-418).

“Operably linked” refers to a first sequence(s) being positioned sufficiently proximal by recombinant DNA technology to a second sequence(s) so that the first sequence(s) can exert influence over the second sequence(s) or a region under control of that second sequence. “Operably linked” according to the present invention means that the heat inducible promoter and the coding region for a protein or peptide are actively linked to each other following recombinant DNA technology and that the pair of said promoter and said coding region does not occur in wild-type species of the host cell (or its location is different) in the linked form according to the present invention. This means that either the protein gene has been introduced 3′ to a wild-type heat inducible promoter or a heat inducible promoter is introduced 5′ to a wild-type coding region or that both heat inducible promoter and the coding region for the protein are introduced in the host cell encapsulated at any (non-wild-type) position. Usually the heat inducible promoter and/or the gene encoding a protein, peptide or functional nucleic acid are exogenous (foreign), i.e. not derived from the wild-type version of the host cell encapsulated.

According to the present invention a “peptide” comprises less than 40 amino acids and a “protein” more than 40 amino acids, without being restricted to a distinct type of peptide or protein.

The capsule according to the present invention contains at least one cell harbouring a nucleic acid molecule, which is integrated or not integrated in the chromosomal DNA of said cell, and which comprises a heat inducible promoter which may be regulated and activated in the presence of heat. The nucleic acid molecule linked 5′ to the promoter encodes for a protein, a peptide or a functional nucleic acid. These transcription and translation products may directly or indirectly act as therapeutic agents (e.g. antibodies, insulin, hGH, EPO, hNGF, CNTF, GDNF, proopiomelanocortin, iNOS, IL-2, endostatin) when secreted from the capsule or they may catalyse the synthesis of another therapeutic substance (e.g. ifosfamide) or produce viral particles infecting neighbouring cells. According to the present invention a functional nucleic acid is intended to be e.g. siRNA, shRNA, miRNA, antisense RNA, ribozymes.

The heat inducible promoter has to be activatable at temperatures between 38° and 50° C., preferably between 40° and 48° C. At the basal body temperature (36° to 37° C.) the promoter should not be active (basal expression). Of course too high temperatures are also not suitable, because normal tissue is subjected to necrosis at temperatures above 44°, depending on the time of exposure. Therefore, the optimal temperature range within which the heat inducible promoter is activated reaches preferably from 41°/42° to 48° C.

Heat inducible promoters have been used in gene therapy for the expression of proteins, active compounds and the like at predefined sites in an individual. For instance, in WO 98/40105 a gene therapeutic method is disclosed, wherein a nucleic acid molecule producing a protein under the control of a heat inducible promoter is introduced into the chromosomal DNA of an individual. In order to activate said promoter a laser, a microwave, radiofrequency or ultrasound source causing the formation of heat at the desired site have been used. In US 2003/0045495 the use of a construct comprising a heat inducible promoter operably linked to a nucleic acid fragment encoding for a therapeutic polypeptide affecting the growth of a tumor is disclosed. In contrast thereto, the capsules according to the present invention can be used without the utilisation of gene therapy, because the heat inducible promoter operably linked to a nucleic acid e.g. encoding for a protein is not introduced in the chromosomal DNA of the treated individual (said nucleic acid molecule will remain in the cells in the capsule).

After introducing the nucleic acid molecule comprising a heat inducible promoter operably linked to a nucleic acid fragment to be transcribed/translated into the cells, a suitable clone may be selected prior to encapsulation. It is an important advantage of encapsulated cells that prior to implantation or administration a clone can be isolated, which features the desired characteristics (e.g. low basal expression, high expression rate upon induction, long-time stability and viability). Furthermore, the optimal inducing conditions (e.g. temperature) can be determined ex vivo.

In order to activate the heat inducible promoter and to induce the transcription of a nucleic acid fragment, which is optionally translated into a protein or peptide, in a more specific manner, localised heat has to be provided to the part of the body or to the tissue where the capsules according to the present invention are present. To allow a localised heating of cells comprising a heat inducible promoter operably linked to a nucleic acid fragment and to reduce at the same time the heating of the surrounding cells, several methods involving invasive and non-invasive method steps may be employed. For instance, an invasive method may employ a catheter, which tip will be heated or which contains an optical guide to direct a laser beam to said cells. To avoid or to reduce surgical interventions to a minimum, non-invasive methods are preferably used. These methods may employ ultrasound (see e.g. WO 98/06864), microwaves, radiofrequency and infrared radiation (see e.g. Samulski T. V., Biomedical Uses of Radiation. W. Hendee (Ed.). VCH Publishers, Weinheim, Germany, pp. 1133-1223 (1999)).

The capsule according to the present invention may be used, for instance, for the treatment of hereditary diseases, particularly diabetes (insulin), dwarfism (hGH), β-thalassaemia (EPO) and haemophilia B (factor XI), cancer (e.g. by inducing the expression of endostatin, IL-2, iNOS), pain (e.g. by inducing the expression of proopiomelanocortin), neurodegenerative diseases (e.g. by inducing the expression of hNGF, CNTF, GDNF). Secreted antibodies and antibody fusion proteins, as well as the application of protein transduction domains like Antennapedia and TAT fragments further extend the list of possible applications. Further applications (comparable to gene therapeutic applications)can be found for instance in Dietz P H G and Bähr M (Mol. Cell. Neurosci. (2004) 27:85-131), Orive G et al. (TIB (2004) 22:87-92), Emery D W (Clin. Appl. Immunol. Rev. (2004) 4:411-422), El-Aneed A (Eur. J. Pharmac. (2004) 498:1-8), Aebischer P and Ridet J L (Trends Neurosc. (2001) 24:533-540) and Günzburg W H (Curr. Opin. Molec. Therap. (2004) 6:258-259).

The definition “heat emitting agent” relates to agents, substances and material compositions capable to emit heat when exposed to electromagnetic radiation, to ultrasound waves, to a magnetic field or to other heat inducing radiations. To guarantee a safe use of a capsule according to the present invention, said heat emitting agent has to be harmless when introduced in an animal or human body. “Heat emitting agents” are known to the person skilled in the art and may be found in several text books and in the patent literature. For instance, U.S. Pat. No. 4,106,488 (particles produce heat by the application of electromagnetic energy), U.S. Pat. No. 6,344,272 and US 2003/0118657 (a laser induces the heat production of nanoparticles coated by a metal layer), U.S. Pat. No. 4,574,782 (ferromagnetic particles are induced to produce heat by the application of a magnetic field), Wust P et al. (The Lancet Oncology (2002), 3:487-497) disclose heat emitting agents and physical parameters inducing an optimal heat emission of said agents (e.g. wavelength of the electromagnetic radiation, frequency of the magnetic field). Although water may in principle also be regarded as a “heat emitting agent”, because e.g. microwaves are able to excite water molecules in order to let water emit heat, agents which have significantly higher absorption characteristics for a given radiation are preferred according to the present invention, because with such external agents a specific heating may be achieved (in contrast to water which is present in each cell/cell surrounding).

Preferably said agent emits heat energy when exposed to an electromagnetic radiation within 300 and 3000 nm, preferably within 400 and 2000 nm, more preferably within 500 and 1500 nm, particularly within 600 and 1400 nm. Agents emitting heat energy at said wavelengths include e.g. nanoshells (U.S. Pat. No. 6,344,272 and US 2003/0118657). When microwaves are applied the wavelength to be applied to induce heat emission of said agent may be selected within the range of 300 μm to 30 cm (frequency 1 GHz to 1 THz).

According to a preferred embodiment of the present invention the heat emitting agent emits heat energy when exposed to a magnetic field with a frequency in the range between 10 kHz and 100 MHz, preferably between 25 kHz and 50 MHz, more preferably between 50 kHz and 20 MHz, in particular between 50 kHz to 2 MHz, with a field strength of 0-30 kA/m, preferably 0-15 kA/m (see e.g. also Pankhurst Q A et al., J. Phys. D: Appl. Phys (2003) 36:R167-R181). If a magnetic field is applied to induce heat emission, the emitting agent has to be at least in part magnetic, particularly ferromagnetic, paramagnetic or superparamagnetic.

According to the present invention also ultrasound may be used to induce the heat emission at a defined area. The use of ultrasound for the control of the expression of therapeutic genes under the control is, for instance, described in Rome C et al. (Methods (2005) 35:188-198). Therein focused ultrasound is used for non-invasive local heating in order to activate hsp70 promoters.

The heat emitting agent preferably comprises at least one particle.

Said particle may be of any geometrical or non-geometrical shape. However, spherical, cubical, cylindrical, hemispherical and elliptical shapes or the like are preferred. The particle(s) may be suspended in a liquid or a gel. Especially particles are suitably employed in a capsule according to the present invention, because they are easy to handle. In order to produce the required temperatures which are needed to activate the heat inducible promoter a certain amount of particles has to be present in the capsules and/or in the tissue in which the capsules are embedded. Therefore, 0.5 to 500 mg, preferably 1 to 200 mg, more preferably 1.5 to 100 mg, particularly 5 to 10 mg, particles, preferably magnetic particles, per cm³ capsules or tissue have to be employed (e.g. Pankhurst Q A et al., J. Phys. D: Appl. Phys (2003) 36:R167-R181).

According to a preferred embodiment of the present invention the particle comprises a metal.

Said metal may be selected from the group consisting of iron, gold, silver, nickel and combinations thereof. The particle according to the present invention may comprise said metal in an elementary form or in form of a substantially insoluble metal compound (e.g. oxide), which preferably does substantially not react under physiological conditions with a liquid comprising water (i.e. body fluid).

The particle is preferably magnetic, preferably ferromagnetic, paramagnetic or superparamagnetic.

Magnetic particles, in particular particles comprising iron, magnetite, maghemite, iron alloys, nickel, nickel alloys, cobalt, cobalt alloys and combinations thereof, may be used as heat emitting agent when a magnetic field is applied on the capsules. Such particles are used in the treatment of cancer by administering said particles e.g. by injection to a patient followed by the application of a magnetic field at the site of the tumor. The magnetic field induces the emission of heat from said particles (see e.g. Pankhurst Q A et al., J. Phys. D: Appl. Phys (2003) 36:R167-R181, U.S. Pat. No. 4,106,488 and U.S. Pat. No. 4,574,782).

The use of magnetic particles in a capsule according to the present invention allows to induce the emission of heat on a specific site by the application of a substantially focused magnetic field. Since the field strength of the magnetic field required to induce the emission of heat is normally in a range which does not harm the health of an individual and since the creation of said magnetic field can be achieved with simple devices, such magnetic particles are preferably employed. Magnetic particles which may be used in capsules according to the present invention are discussed, for instance, in Pankhurst Q A et al. (J. Phys. D: Appl. Phys (2003) 36:R167-R181).

The particle is preferably between 1 nm to 100 μm, preferably between 2 nm to 50 μm, more preferably between 5 nm to 20 μm, particularly between 10 nm to 15 μm, in diameter.

Although prokaryotic cells may be employed in a capsule according to the present invention, the cell is preferably a eukaryotic cell.

Since the capsules according to the present invention are preferably employed in human and animal hosts the cells are preferably eukaryotic cells. The use of eukaryotic cells guarantees the compatibility of said cells and the products (proteins, peptides, nucleic acids or other therapeutic agents) secreted by said cells with the host and reduces the risk of immune rejection of said cells and the metabolic products produced from said cells. Therefore, the cells are selected correspondingly. However, also prokaryotic cells may be used in capsules according to the present invention (see e.g. Chang T M S, Nature Reviews Drug Discovery (2005) 4:221-235).

The at least one cell is preferably a mammalian cell, in particular a human or an animal cell. According to the present invention all human or animal cells known in the state of the art and which may be suited to recombinantly produce a protein, polypeptide or peptide or a functional nucleic acid may be employed.

At least one recombinant cell may be selected from the group consisting of BHK, 293, NIH 3T3, Neuro2A, immortalised human fibroblasts or myoblasts, Lactobacillus delbrueckii, Escherichia coli, Klebsiella aerogenes and combinations thereof.

According to the present invention all known heat inducible promoters or heat responsive elements in a heat shock gene may be employed in a cell comprising the recombinant nucleic acid molecule as described above.

According to another preferred embodiment of the present invention the heat inducible promoter is selected from the group consisting of hsp70, hsp20-30, hsp27, hsp40, hsp60, hsp90 and combinations thereof.

The heat inducible promoter is preferably a hybrid or chimeric heat inducible promoter.

Genetic engineering allows the construction of hybrid or chimeric promoters which may have enhanced effects in comparison to wild type promoters. For instance, it is possible to combine a heat inducible promoter with other elements, which may enhance the mRNA translation, or with other promoters or parts thereof, which are responsive to non-heat stimuli (e.g. to chemicals).

According to a preferred embodiment of the present invention the hybrid or chimeric heat inducible promoter comprises a minimal promoter and at least one regulatory element of a heat inducible promoter.

Such promoters, for instance, are disclosed in Bajoghli B. et al. (Dev Biol. (2004) 271:416-30). Heat inducible promoters comprise so-called heat shock elements. With genetic engineering these elements can be multimerised resulting in a promoter with a multiplicity of said elements. Single or “multimerised” heat shock elements can be fused to other promoters in order to get hybrid or chimeric heat inducible promoters.

The heat inducible promoter is preferably a promoter as described in Austrian patent application A 674/2004. Therefore, the heat inducible promoter according to the present invention comprises preferably a DNA stretch which is characterised in that it comprises at least 2 consensus sequences, each consensus sequence consisting of 3 pentameric units, said pentameric units having a sequence XGAAY or an inverse sequence Y′TTCX′, X being selected from the group consisting of A, T, G, and C, and Y of at least one, preferably two, still preferred all three, of said 3 pentameric units of at least one consensus sequence being selected from the group consisting of A, T, and C, the Y of the remaining pentameric units of said at least one consensus sequence being selected from the group consisting of A, T, G, and C, whereby in the case that said DNA stretch comprises more than 6 consensus sequences, Y of all pentameric units is selected from the group consisting of A, T, G, and C. This DNA stretch has shown to be optimal in expression induction with low background activity, high inducibility and lack of tissue specific expression.

With respect to the inverse sequence “X′” relates to a nucleotide being complementary to the “X” of the non-inverse pentameric unit. This means that “X′” is selected from the group consisting of A, T, G and C. The “Y′” which is complementary to the “Y” of the non-inverse pentameric unit is therefore selected from the group consisting of T, A and G for at least one, preferably two, still preferred all, pentameric units of at least one consensus sequence, whereby the “Y′” of the remaining pentameric units is selected from the group consisting of A, T, G and C. Therefore, in the DNA stretch at least one pentameric unit, be it the inverse or non-inverse sequence, comprises either an Y being selected from A, T and C or an Y′ being selected from A, T and G. It has been shown that in the case that the DNA stretch comprises a lower number of consensus sequences, for example two to six consensus sequences, it is important that the consensus sequence shows optimal inducibility which is the case when Y is not a G or Y′ is not a C. However, in the case that the DNA stretch comprises a larger number of consensus sequences, e.g. more than six consensus sequences, the Y or Y′ may be selected from the group consisting of A, T, G and C, since the higher number of consensus sequences causes protein expression induction with superior properties. In other words: the lower the numbers of consensus sequences in the DNA stretch, the more it is important to provide an optimal pentameric unit which is the case, when Y is not G and Y′ is not C.

It is possible that one consensus sequence comprises only non-inverse pentameric units XGAAY or only inverse pentameric units Y′TTCX′. However, it is also possible that one consensus sequence comprises two non-inverse pentameric units and one inverse pentameric unit or one non-inverse pentameric unit and two inverse pentameric units. One consensus sequence may comprise identical pentameric units with respect to the X/X′ and Y/Y′. However, in one consensus sequence 2 or all 3 pentameric units may vary in the the X/X′ and Y/Y′.

Said DNA stretch may further comprise identical consensus sequences or non-identical consensus sequences or, in the case that there are three or more consensus sequences in the DNA stretch, two or more consensus sequences can be identical and the remaining consensus sequences different. The difference can be either with respect to the selection of the X and/or Y (Y′ and/or X′) or with respect to the presence of non-inverse and inverse sequences or both.

The preferred DNA stretch should comprise at least two consensus sequences. However, the DNA stretch may comprise more than 10, more than 20, more than 30, more than 40 or more than 50 consensus sequences. Furthermore, the DNA stretch may comprise additional sequences, sequence fragments or single nucleic acids which may be of any specific or non-specific sequence or even an additional pentameric unit. For example the DNA stretch may comprise 2 consensus sequences and an additional 1 or 2 pentameric units.

Preferably, the DNA stretch comprises 4-24, preferably 7-16, still preferred 8 consensus sequences. It was shown that these numbers of consensus sequences are optimal, since on the one hand the DNA stretch comprises a sufficient number of consensus sequences in order to show strong inducibility and on the other hand the DNA stretch is not too long to show negative side activities, like recombination and others.

Advantageously, the consensus sequences are separated by 2 to 10 bp, preferably by alternatingly 3 and 6 bp. It was found that the respective factor, e.g. heat shock factor, binds in an optimal manner, when the consensus sequences are not directly linked to one another. These short spacer sequences allow for specific binding and activation of the respective factor to each consensus sequence.

According to a preferred embodiment the middle pentameric unit of at least one, preferably each consensus sequence is an inverse sequence compared to the outer pentameric units, preferably sequence Y′TTCX′. This means that the middle pentameric unit may be the non-inverse or the inverse sequence, depending on whether the two outer sequences are inverse or non-inverse. By alternatingly providing a non-inverse and inverse sequence the respective factor binds strongly and shows high inducibility, whereby it is shown to be optimal when at least one, preferably each consensus sequence is as follows: XGAAY Y′TTCX′ XGAAY (SEQ ID NO: 1; note that the Sequence Listing submitted herewith contains “N” in place of “X,” because the Patent-In program employed to create the Sequence Listing defines “X” as used in the Sequence Listing in a manner different from that used in this specification. In this specification and claims, “X,” unless further defined in a specific location, denotes any nucleotide).

Advantageously, the X is C or G, still preferred A. In the case that X is a C or G, the respective factor shows excellent binding and activating properties, which are, however, even better in the case that X is an A. Accordingly, X′ is preferably G or C and still preferred T. This applies for at least one X of the whole DNA stretch, preferably several X of the DNA stretch, still preferred all X of the DNA stretch. A DNA stretch comprising pentameric units in which X is always A therefore shows ideal properties.

In a further advantageous DNA stretch Y is C. Accordingly, for the inverse sequence Y′ is preferably G. As mentioned above for X, this applies for at least one Y of the whole DNA stretch, preferably several Y of the DNA stretch, still preferred all Y of the DNA stretch. Therefore, a DNA stretch, in which all Y are a C shows optimal inducibility.

Advantageously, therefore at least 1, preferably all consensus sequences are AGAAC GTTCT AGAAC (SEQ ID NO: 2). As already mentioned above, in the case that the DNA stretch comprises 6 or less consensus sequences, it is preferable that all consensus sequences are as defined above. In the case that the DNA stretch shows more than 6 consensus sequences, it is possible that 1 or more pentameric units show the above mentioned variations of X or Y or the respective X′ or Y′, however, with similarly high performances.

Another aspect of the present invention relates to an implant comprising at least one capsule according to the present invention.

The capsules according to the present invention may be used for the manufacture of an implant which can be used e.g. in cell therapy. In order to allow proteins, peptides, functional nucleic acids or virus particles (see e.g. U.S. Pat. No. 6,776,985) produced by the cells in the capsules to pass the outer barrier of the implant, said implant may comprise a permeable material of suitable pore size. Implants composed of encapsulated cells are known to a person skilled in the art and already employed for the treatment of several diseases (see e.g. Chang T. M., Ann NY Acad Sci (1999), 875:71-83).

“At least one capsule” refers to the fact that the implant itself may be a capsule according to the present invention. Therefore, if the implant comprises only one capsule, said capsule is intended to be an implant.

The implants according to the present invention can also be used to treat a wide range of diseases. Advantageously these implants comprise cells whose expression machinery can simply be regulated by changing the temperature or cell stress within said implant. Said changing can be controlled by exposing said implant to electromagnetic radiation or to a magnetic field which induces a heat emitting agent present in said implant to emit heat. Such implants have many advantages in several areas of application enabling a patient to control the release of a therapeutic agent on his own simply by exposing the implant to electromagnetic radiations or to a magnetic field. For instance, an implant comprising a cell capable to produce and to secrete insulin into a tissue or into the bloodstream when exposed to heat may be used for patients suffering from diabetes (a device measuring the insulin concentration in the blood and then generating a magnetic field or electromagnetic radiation of defined intensity).

Another aspect of the present invention relates to a kit comprising
a vector or nucleic acid molecule comprising a heat inducible promoter operably linkable to a nucleic acid encoding for a protein, a peptide or a functional nucleic acid,
at least one heat emitting agent as defined above, and
optionally a cell capable to express a protein or a peptide or to transcribe a functional nucleic acid molecule under the control of a heat inducible promoter, which may be used to prepare a capsule or an implant as defined above.

A nucleic acid fragment encoding for a protein, a peptide, a functional nucleic acid or a virus particle may be introduced by genetic engineering into the vector or the nucleic acid molecule of the kit. Afterwards the resulting construct may be transferred into a cell capable to express a protein (e.g. a virus particle, an antibody) or a peptide or to transcribe a functional nucleic acid molecule (e.g. siRNA, virus particle) under the control of a heat inducible promoter, which may be part of the kit. The cell harbouring the exogenous nucleic acid structure integrated or non-integrated in its chromosome may then be encapsulated with at least one heat emitting agent of the kit.

Another aspect of the present invention relates to a medicament or pharmaceutical composition comprising at least one capsule as defined above.

The medicament and the pharmaceutical composition according to the present invention may be prepared for intrapulmonary, mucosal, oral, intravenous, subcutaneous or intramuscular administration. This may allow delivering the capsules to a desired site in a human or animal body. At said site the capsules may be treated with electromagnetic radiation or a magnetic field to induce the generation of heat within the capsules and hence to activate the heat inducible promoter. Therefore, the pharmaceutical composition may preferably be used, for instance, to treat diseases affecting the gut or the stomach.

Another aspect of the present invention relates to a method for the manufacture of a capsule or an implant according to the present invention comprising the steps:
providing a cell capable to recombinantly produce a protein, a peptide or a functional nucleic acid under the control of a heat inducible promoter according to the present invention,
transferring a nucleic acid molecule comprising a heat inducible promoter as defined above operably linked to a nucleic acid encoding for a protein, a peptide or a functional nucleic acid, and
encapsulating said cell together with a heat emitting agent as defined herein in a permeable membrane.

The present invention is further illustrated by the following examples, without being restricted thereto.

### EXAMPLES:

### Example 1

An artificial heat sensitive promoter (Bajoghli B. et al. Dev Biol. (2004) 271:416-30; Austrian patent application A 674/2004), comprising 8 idealised heat shock elements (HSE) and a minimal promoter were used to generate a construct driving the marker gene Gfp. Due to the perfect symmetry of the HSEs, a second minimal promoter was used upstream of this promoter in opposite orientation to activate another marker gene, firefly luciferase. In transient cell culture experiments, this construct bidirectionally activates both marker genes in a heat sensitive manner (Bajoghli B. et al. Dev Biol. (2004) 271:416-30). Human HeLa cells were transiently transfected using PEI (polyethelene imine) and were then encapsulated according to the following protocol (Löhr et al. (1998) Gene Ther 5:1070-78). 1×10⁷ cells were suspended in 1 ml PBS containing 4% cellulose phosphate and 5% FCS. Using an encapsulator from Inotech (Dottikon, Switzerland) the suspension was allowed to drop in a regulated manner into a precipitation bath containing 3% polydiallyldimethyl ammonium in PBS where capsules of 200-500 μm formed. The capsules were washed twice with medium and were then taken into tissue culture.

The encapsulated cells incubated in cell culture medium were then subjected to a temperature of 43° C. for 2 hours and then returned to normal cell culture conditions (37° C.). On the next day the encapsulated cells were analysed for Gfp-activity under the fluorescence microscope and luciferase activity was measured with a luminometer assay. Both marker genes were strongly activated after heat treatment, whereas no activation could be observed for untreated cells. The effects were comparable to non-encapsulated cells kept in parallel, thus demonstrating that the encapsulation does not affect activation of the artificial HSE promoter construct.

More reproducible results can be obtained from stably integrated reporter constructs. HeLa cells were therefore co-transfected with the promoter construct and a puromycin resistance plasmid. After puromycin treatment for 2 weeks, Gfp positive (after heat treatment) cell clones were selected. The clone with the lowest background activity was then used for encapsulation. After heat activation all cells uniformly showed effects comparable to transiently transfected cells.

### Example 2

In order to establish conditions for the hyperthermia treatment, increasing intensities of an oscillating magnetic field were subjected to cells encapsulated together with magnetic particles. For this purpose 10 mg / magnetite (Fe₃O₄) magnetic particles of 10 μm average size were mixed with 1 ml cell suspension prior to encapsulation. For generation of the oscillating magnetic field, a coil of 7 cm length and 7 cm diameter was activated at 118 kHz with a field strength of 0-30 kA/m. The encapsulated cells in the cell culture medium were treated with varying intensities of the magnetic field for 1 hour. Thereafter the cells were analysed with methylene blue for occurrence of cell death. Since temperatures exceeding 43° C. may result in increasing cell death (depending on the type of cell used), this experiment was used to roughly determine the critical field strength, assuming that the amount of cell death reflects the temperature inside the capsules.

### Example 3

In another experiment, the stable HSE HeLa cell line was used for encapsulation and activated according to the results of the previous experiment. Indeed high activity of marker gene activation could be observed, counteracted by increasing cell death at higher intensities. This experiment was repeated several times, with variations in the amount of magnetic particles added and negative controls containing no particles. Taken together, these experiments clearly demonstrated that a heat sensitive promoter can be activated within capsules by magnetic particles in an oscillating magnetic field. The amount of magnetic particles added, directly affected the field strength necessary to obtain maximum promoter activity. Most importantly, for defined reaction conditions applied for encapsulation, the reporter construct was reproducibly activated at the same field strength.

### Example 4

Finally a mixing experiment was performed with capsules containing either normal HeLa cells or capsules containing the stable HSE reporter cells. Magnetic particles were added only to one kind of capsules. In a mixture, the different capsules were activated with a magnetic field as established before. In case the particles were present in the capsules containing the reporter cell line, marker gene activation took place as expected. In the opposite case, when the capsules containing the normal cells were heat treated, no activation was observed for the reporter construct, being present in the neighbouring capsules. This demonstrated that a localised temperature increase within the capsules containing the magnetic particles does not significantly affect the neighbouring capsules via the bulk temperature of the cell culture medium.

1.-22. (canceled)

23. A permeable capsule comprising:
at least one cell comprising a recombinant nucleic acid molecule with a heat inducible promoter operably linked to a nucleic acid encoding for a protein, a peptide, or a functional nucleic acid molecule; and
at least one heat emitting agent able to emit heat when exposed to electromagnetic radiation or to a magnetic field.

24. The capsule of claim 23, wherein the electromagnetic radiation comprises radio waves, microwaves, or infrared radiation.

25. The capsule of claim 23, wherein the agent comprises at least one particle.

26. The capsule of claim 25, wherein the particle comprises a metal.

27. The capsule of claim 25, wherein the particle is magnetic.

28. The capsule of claim 27, wherein the particle is ferromagnetic, paramagnetic or superparamagnetic.

29. The capsule of claim 25, wherein the particle is between 1 nm to 100 μm in diameter.

30. The capsule of claim 29, wherein the particle is between 2 nm to 50 μm in diameter.

31. The capsule of claim 30, wherein the particle is between 5 nm to 20 μm in diameter.

32. The capsule of claim 23, wherein the at least one cell is a eukaryotic or a prokaryotic cell.

33. The capsule of claim 23, wherein the at least one cell is a mammalian cell.

34. The capsule of claim 33, wherein the at least one cell is a human or an animal cell.

35. The capsule of claim 23, wherein the heat inducible promoter comprises hsp70, hsp20-30, hsp27, hsp40, hsp60, hsp9, or a combination thereof.

36. The capsule of claim 23, wherein the heat inducible promoter is a hybrid or chimeric heat inducible promoter.

37. The capsule of claim 36, wherein the hybrid or chimeric heat inducible promoter comprises a minimal promoter and at least one regulatory element of a heat inducible promoter.

38. The capsule of claim 23, wherein the heat inducible promoter comprises at least 2 consensus sequences, each consensus sequence consisting of 3 pentameric units, the pentameric units having a sequence XGAAY or an inverse sequence Y′TTCX′, wherein X is A, T, G, or C, and Y of at least one of the 3 pentameric units of at least one consensus sequence is of A, T, or C, Y of the remaining pentameric units of the at least one consensus sequence is A, T, G, or C, wherein if the DNA molecule comprises more than 6 consensus sequences, Y of all pentameric units is A, T, G, or C.

39. The capsule of claim 38, wherein Y of at least two of the 3 pentameric units of at least one consensus sequence is A, T, or C.

40. The capsule of claim 39, wherein Y of all three of the 3 pentameric units of at least one consensus sequence is A, T, or C.

41. The capsule of claim 38, wherein the promoter comprises 4 to 24 consensus sequences.

42. The capsule of claim 41, wherein the promoter comprises 7 to 16 consensus sequences.

43. The capsule of claim 42, wherein the promoter comprises 8 consensus sequences.

44. The capsule of claim 38, wherein the consensus sequences are separated by 2 to 10 bp.

45. The capsule of claim 44, wherein the consensus sequences are alternatingly separated by 3 and 6 bp.

46. The capsule of claim 38, wherein the middle pentameric unit of at least one consensus sequence is an inverse sequence compared to the outer pentameric units of that consensus unit.

47. The capsule of claim 46, wherein the middle pentameric unit of each consensus sequence is an inverse sequence compared to the outer pentameric units of such consensus sequence.

48. The capsule of claim 46, wherein the middle pentameric unit is of sequence Y′TTCX′.

49. The capsule of claim 38, wherein X is A, C, or G.

50. The capsule of claim 49, wherein X is A.

51. The capsule of claim 38, wherein Y is C.

52. The capsule of claim 38, wherein at least one of the consensus sequences is AGAAC GTTCT AGAAC.

53. The capsule of claim 52, wherein all consensus sequences are AGAAC GTTCT AGAAC.

54. The capsule of claim 23, further defined as comprised in a medicament or pharmaceutical composition.

55. The capsule of claim 23, further defined as comprised in an implant.

56. A kit comprising:
a vector or nucleic acid molecule comprising a heat inducible promoter operably linkable to a nucleic acid encoding for a protein, a peptide or a functional nucleic acid; and
at least one heat emitting agent as defined in claim 23.

57. The kit of claim 56, further defined as comprising a cell able of expressing a protein or a peptide or transcribing a functional nucleic acid molecule under the control of a heat inducible promoter.

58. A method for the manufacture of a capsule of claim 23 and/or of an implant of claim 55 comprising:
providing a cell capable to recombinantly produce a protein, a peptide, or a functional nucleic acid under the control of a heat inducible promoter;
transferring to the cell a nucleic acid molecule encoding for a protein, a peptide or a functional nucleic acid molecule, the nucleic acid molecule operably linked to a heat inducible promoter; and
encapsulating the cell together with a heat emitting agent that can emit heat when exposed to electromagnetic radiation or to a magnetic field in a permeable membrane.

59. A method of delivering a nucleic acid molecule encoding for a protein, a peptide or a functional nucleic acid molecule to a location comprising:
obtaining a permeable capsule comprising:
at least one cell comprising a recombinant nucleic acid molecule with a heat inducible promoter operably linked to a nucleic acid encoding for a protein, a peptide, or a functional nucleic acid molecule; and
at least one heat emitting agent able to emit heat when exposed to electromagnetic radiation or to a magnetic field;
providing the capsule to the location; and
exposing the capsule to electromagnetic radiation or to a magnetic field;
wherein the nucleic acid molecule is delivered to the location.

60. The method of claim 59, further defined as a method of providing gene therapy to a human or non-human animal subject.
